(19) United States
(12) Patent Application Publication
(10) Pub. No.: US 2016/0108002 A1
(43) Pub. Date: Apr. 21, 2016

(54) UNIVERSAL READER MOLECULE FOR RECOGNITION TUNNELING

(71) Applicant: ARIZONA BOARD OF REGENTS acting for and on behalf of ARIZONA STATE UNIVERSITY, Scottsdale, AZ (US)

(72) Inventors: Peiming ZHANG, Gilbert, AZ (US); Stuart LINDSAY, Phoenix, AZ (US); Sovan BISWAS, Tempe, AZ (US); Suman SEN, Tempe, AZ (US)

(21) Appl. No.: 14/894,782

(22) PCT Filed: May 30, 2014
(86) PCT No.: PCT/US14/40323

§ 371 Date: Nov. 30, 2015

Related U.S. Application Data
(60) Provisional application No. 61/829,229, filed on May 30, 2013.

(51) Int. Cl. C07D 235/24; G01N 33/487

(52) U.S. Cl. CPC C07D 235/24; G01N 33/48721

(57) ABSTRACT

Some embodiments of the present disclosure are directed to a compound 5(6)-mercapto-1H-benzo[d]imidazole-2-carboxamide (“BIA”) which yields enhanced signals for recognition tunneling. Other embodiments are directed toward methods for producing such compounds as well as apparatuses and systems which utilize such compounds for recognition tunneling for molecule identification/sequencing (for example).

PRIORITY

This application claims priority to U.S. provisional application No. 61/829,229 titled “UNIVERSAL READER MOLECULE FOR RECOGNITION TUNNELING”, filed on May 30, 2013, the disclosure of which is incorporated herein by reference in its entirety.

### STATEMENT REGARDING FEDERALLY SPONSORED RESEARCH & DEVELOPMENT

Embodiments of this disclosure were made with government support under NIH Grant No. HG006323, awarded by the National Institute of Health. The U.S. Government has certain rights in inventions disclosed herein.

### BACKGROUND

This disclosure is related to a previous series of disclosures on a readout system for nucleic acid sequences, for example (WO2008/124706A2, WO2009/117517, WO2009/117522A2, WO2010/042514A1, WO2011/097171, 61/300,678, and 61/620,167), and peptide sequences (U.S. provisional patent application Nos. 61/593,552, and 61/647,847), based on the distinct tunneling signals generated when an analyte is trapped by reading molecules chemically tethered to two closely spaced electrodes via a mechanism called “Recognition Tunneling”. In some of these earlier disclosures, a molecule that can be used as a universal reader for DNA bases and many amino acids and sugars is disclosed: 4(5)-(2-mercaptoethyl)-1H-imidazole-2-carboxamide (see FIG. 1). This molecule contains a heterocycle and a carboxamide-both providing hydrogen bonding donors and acceptors, and it is attached to the metal substrates by means of the two carbon (ethylene) linker terminated with thiol—which forms the attachment bond to the metal. The ethylene linker leads to significant attenuation of electronic signals, which are better transmitted by π conjugated (aromatic) molecules. In addition, 4(5)-(2-mercaptoethyl)-1H-imidazole-2-carboxamide has proven problematic when the target molecule has a hydrophobic character (e.g., like tyrosine or tryptophan).

Accordingly, what is desired is a reader molecule that introduces less attenuation of the tunneling signal, and that adds hydrophobic character to the reader molecules. These objects are achieved in the reader molecule according to some embodiments of the present disclosure.

### SUMMARY OF SOME OF THE EMBODIMENTS

In some embodiments, a molecule 5(6)-mercapto-1H-benzo[d]imidazole-2-carboxamide (also referred to as 5(6)-mercapto-1H-benzimidazole-2-carboxamide, or “BIA”) is disclosed, which in some embodiments, yields enhanced signals for recognition tunneling.

Such embodiments, as well as other embodiments of the present disclosure, are detailed below, with at least some of the supporting subject matter for some of the embodiments being found in the attached drawings, a brief description of which is provided below.

### BRIEF DESCRIPTION OF THE FIGURES

FIG. 1 is an illustration of the chemical structure of 4(5)-(2-mercaptoethyl)-1H-imideazole-2-carboxamide.

FIG. 2 is an exemplary illustration of the calculated structure of two BIA molecules as tethered to electrodes and trapping a deoxyadenosine molecule.

FIG. 3 is an exemplary illustration of the synthesis of 5(6)-mercapto-1H-benzo[d]imidazole-2-carboxamide according to some embodiments.

FIG. 4 is an exemplary STM (scanning tunneling microscope) image of a palladium substrate after functionalization, according to some embodiments.

FIGS. 5A and 5B are plots of tunneling spectra of deoxyadenosine monophosphate, according to some embodiments.

FIG. 6 is a plot of tunneling spectra of deoxycytidine monophosphate having two level signals as shown in FIGS. 5A and 5B.

FIG. 7 is a plot of tunneling spectra of deoxyguanosine monophosphate.

FIG. 8 is a plot of a tunneling spectra of thymidine monophosphate.

FIGS. 9A, 9B is a plot of a tunneling spectra of deoxy-5-methylcytidine monophosphate having two level signals.

FIG. 10 is an exemplary image of a tunneling spectra of glycine.

FIGS. 11A-11D are plots illustrating the results of SVM analysis for imidazole (FIGS. 11A, 11C) and for benzimidazole (FIGS. 11B, 11D) at setpoints of 2 pA (FIGS. 11A, 11B) and 4 pA (FIGS. 11C, 11D), respectively.

### DETAILED DESCRIPTION OF SOME OF THE EMBODIMENTS

This disclosure is related to PCT Application Nos. WO2008/124706A2, WO2009/117517, WO2009/117522A2, WO2010/042514A1, and WO2011/097171; and to U.S. Provisional Application Nos. 61/300,678, 61/620,167, 61/593,552, and 61/647,847, the disclosure of each being incorporated herein by reference in its entirety.

Before some embodiments of the present disclosure are described in detail, it is to be understood that such embodiments are not limited to particular variations set forth and may, of course, vary. Various changes may be made to embodiments described and equivalents may be substituted without departing from the true spirit and scope of inventions disclosed herein. In addition, many modifications may be made to adapt a particular situation, material, composition of matter, process, process act(s) or step(s), to the objective(s), spirit or scope of the present disclosure. All such modifications are intended to be within the scope of any and all claims supported by the present disclosure.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

Unless stated otherwise, the term “mixture” as used herein can be used to denote the result of any treatment step described herein. The treatment step can be chemical, physical, or a combination thereof. Accordingly, unless explicitly stated otherwise, a treatment step can act on a mixture of a previous treatment step to yield a new mixture, which can serve as input into the next treatment step, and so on.

The term “about”, when used herein in connection with a numerical indication, is used to indicate a value within 10 percent of the numerical indication. For example, “about 1” can include values ranging from 0.9 to 1.1.

The terms “reader”, “reading molecule”, “reading compound”, “trapping molecule”, “trapping compound”, and variants thereof, as used herein, and refer to a molecule/compound capable of being functionalized to an electrode of a recognition tunneling apparatus, such that during use, the molecule/compound can interact with an analyte passing in proximity to the electrode to form a molecular circuit.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within embodiments of the disclosure. Also, it is contemplated that any optional feature of one and/or another of the disclosed embodiments described herein may be set forth and claimed independently, or in combination with any one or more of the features described herein.

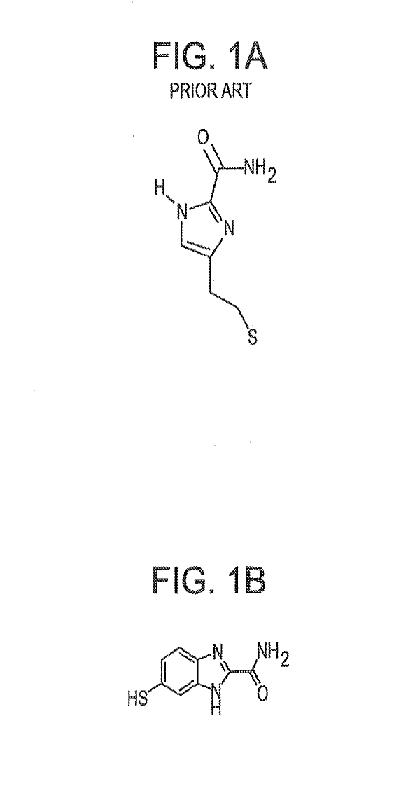

In some embodiments, aspects of the disclosure are directed to a trapping molecule/compound comprising a five membered aromatic ring fused with another ring moiety. In some embodiments, the ring moiety is a conductive ring moiety, or a derivative of a conductive ring moiety, thereby enhancing the conductivity of the compound. In some embodiments, as best illustrated in FIG. 1A, the trapping molecule is 5(6)-mercapto-1H-benzo[d]imidazole-2-carboxamide (“BIA”), having the chemical formula C₈H₇N₃OS. In some embodiments, BIA can be usable for trapping an analyte in a tunnel junction.

In some embodiments, aspects of the disclosure are directed to compositions that include a trapping molecule/compound comprising a five membered aromatic ring fused with another ring moiety. In some embodiments, the ring moiety is a conductive ring moiety, or a derivative of a conductive ring moiety, thereby enhancing the conductivity of the compound. In some embodiments, as best illustrated in FIG. 1A, the trapping molecule/compound is 5(6)-mercapto-1H-benzo[d]imidazole-2-carboxamide (“BIA”). In some embodiments, BIA can be usable for trapping an analyte in a tunnel junction.

In some embodiments, the compositions including the trapping molecule/compound can further include one or more components, such as a solvent for example.

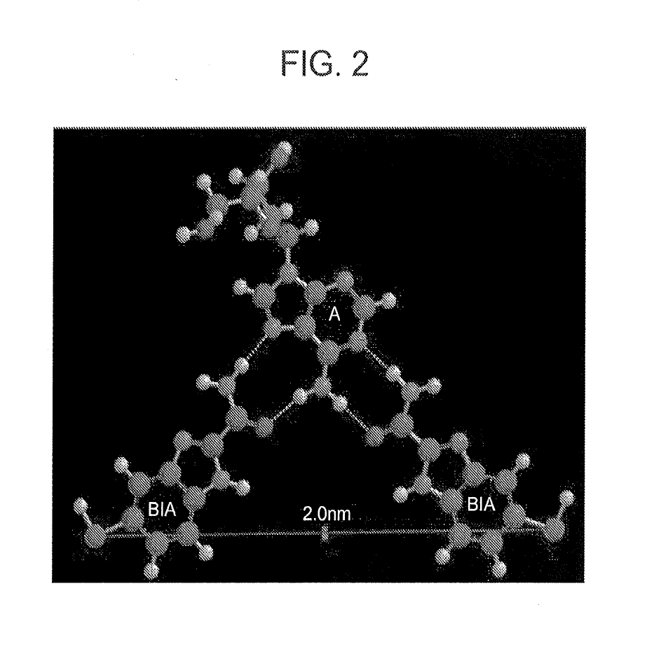

In some embodiments, aspects of the disclosure are directed to a reader molecule/compound comprising a fusion of an aromatic ring with a heterocycle. In some embodiments, the reader molecule/compound forms complexes with at least one of nucleobases, amino acids, other biochemical molecules capable of forming the complexes, and/or the like, through non-covalent interaction(s). In some embodiments, the interaction(s) include at least one of hydrogen bonding, aromatic interactions, stacking interaction(s), hydrophobic interaction(s), and/or the like. In some embodiments, the reader molecule/compound is usable in a recognition tunneling apparatus. In such embodiments, as a result of the formed complexes during use, tunneling current signals are generated in the recognition tunneling apparatus. In some embodiments, the reader molecule is BIA. FIG. 2 illustrates an exemplary embodiment where two BIA molecules interact with the nucleobase adenine (A) through hydrogen bonds so that electrons can tunnel through a nanogap and generate electrical signals for identification of the analyte in a recognition tunneling apparatus, as described later.

In some embodiments, aspects of the disclosure are directed to compositions that include a reader molecule/compound comprising a fusion of an aromatic ring with a heterocycle. In some embodiments, the reader molecule/compound forms complexes with at least one of nucleobases, amino acids, other biochemical molecules capable of forming the complexes, and/or the like, through non-covalent interaction(s). In some embodiments, the interaction(s) include at least one of hydrogen bonding, aromatic interactions, stacking interaction(s), hydrophobic interaction(s), and/or the like. In some embodiments, the reader molecule is usable in a recognition tunneling apparatus. In such embodiments, as a result of the formed complexes during use, tunneling current signals are generated in the recognition tunneling apparatus. In some embodiments, the reader molecule is BIA. FIG. 2 illustrates an exemplary embodiment where two BIA molecules interact with the nucleobase adenine (A) through hydrogen bonds so that electrons can tunnel through the nanogap and generate electrical signals for identification of the analyte.

In some embodiments, the compositions including the reader molecule/compound can further include one or more components, such as a solvent for example.

In some embodiments, a method of synthesizing the trapping molecule and/or the reader molecule described above includes forming a doubly amintated phenol ring, and treating the doubly amintated phenol ring with chloroacetamide. As a result of the treating step, a fusion of an aromatic ring with a heterocycle is formed as the trapping molecule and/or the reader molecule. In some embodiments, the formed trapping molecule and/or reader molecule is BIA.

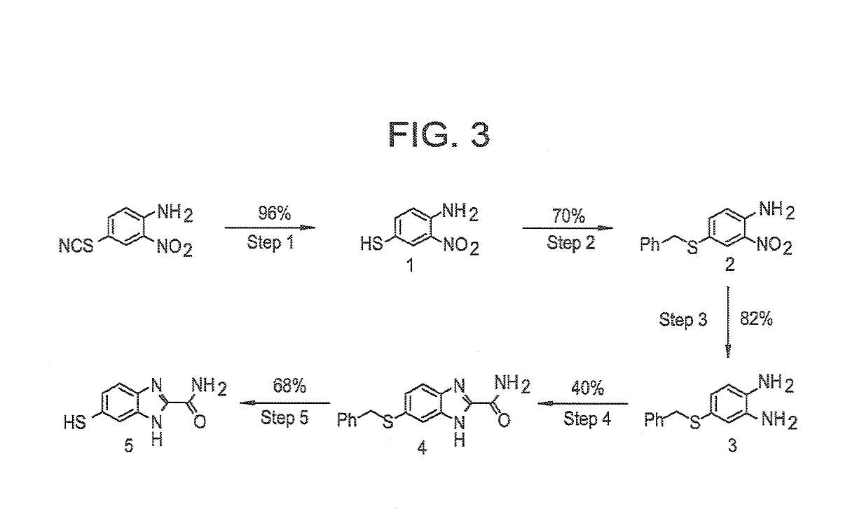

FIG. 3 illustrates an exemplary method for synthesizing BIA, as described in more detail later (see Examples). Generally, in some embodiments, a method for synthesizing BIA includes adding 2-Nitro-4-thiocyanatoaniline in portions to a stirred solution of potassium hydroxide in ethanol at a first temperature. In some embodiments the first temperature is about 2° C., about 3° C., about 4° C., about 5° C., about 6° C., about 7° C., about 8° C., about 9° C., about 10° C., about 12° C., about 13° C., about 15° C., and all values in between.

The method further includes stirring the mixture for a first period of time at a second temperature. In some embodiments, the first period of time is about 20 minutes, about 25 minutes, about 28 minutes, about 30 minutes, about 32 minutes, about 35 minutes, about 40 minutes, and all values in between. In some embodiments, the second temperature is room temperature. In some embodiments, the second temperature is about 20° C., about 22° C., about 25° C., about 27° C., about 29° C., about 30° C., and all values in between.

The method further includes adding an aqueous solution of a strong acid, such as sulfuric acid for example, to the mixture until the color of the mixture changes from a first color to a second color. The method further includes removing one or more solvents from the mixture. The method further includes adding water to the mixture and then extracting water with one or more of ethyl acetate, chloroform, and ethyl ether, to produce an organic extract from the mixture. The method further includes washing the organic extract with brine or water, and drying the washed organic extract over sodium sulfate or magnesium sulfate. The method further includes filtering the mixture and removing the solvent, wherein a red solid is produced.

In some embodiments, the method further includes forming a solution of the red solid/mixture in an organic solvent such as, but not limited to, dichloromethane, tetrahydrofuran (THF), dimethylformamide (DMF), and/or the like. In some embodiments, the method further includes adding into the solution triethylamine, sodium hydroxide, or sodium hydride. In some embodiments, the method further includes adding triethylamine into the solution. In some embodiments, the triethylamine is added dropwise.

In some embodiments, the method further includes stirring the solution for a second period of time at a third temperature. In some embodiments, the second period of time is about 20 minutes, about 25 minutes, about 28 minutes, about 30 minutes, about 32 minutes, about 35 minutes, about 40 minutes, and all values in between. In some embodiments, the third temperature is room temperature. In some embodiments, the third temperature is about 20° C., about 22° C., about 25° C., about 27° C., about 29° C., about 30° C., and all values in between.

In some embodiments, the method further includes adding to the mixture, benzyl bromide, benzyl chloride or benzyl iodide. In some embodiments, the method further includes adding benzyl bromide to the mixture. In some embodiments, the method further includes stirring the mixture for a third period of time at a fourth temperature. In some embodiments, the third period of time is about 25 hours, about 30 hours, about 35 hours, about 40 hours, about 41 hours, about 42 hours, about 43 hours, about 44 hours, about 45 hours, about 50 hours, and all values in between. In some embodiments, the fourth temperature is room temperature. In some embodiments, the fourth temperature is about 20° C., about 22° C., about 25° C., about 27° C., about 29° C., about 30° C., and all values in between.

In some embodiments, the method further includes removing solvent from the mixture, resulting in a crude mixture In some embodiments, the method further includes dissolving the crude mixture in dichloromethane (CH₂Cl₂) or ethyl acetate and washing the dissolved crude mixture with at least one of saturated sodium bicarbonate solution and brine. In some embodiments, the method further includes drying the washed crude mixture over magnesium sulphate (MgSO₄). In some embodiments, the method further includes filtering the dried crude mixture. In some embodiments, the method further includes concentrating the dried crude mixture. In some embodiments, the concentrating is performed via rotary evaporation and/or distillation.

In some embodiments, the method further includes purifying the dried crude mixture In some embodiments, the purifying is performed via flash column chromatography. In some embodiments, the purified product includes 4-(Benzylthio)-2-nitrobenzenamine

In some embodiments, the method further includes dissolving the purified product (e.g., 4-(Benzylthio)-2-nitrobenzenamine) in aqueous ethanol. In some embodiments, the method further includes adding sodium dithionite in portions over a fourth time period. In some embodiments, the fourth time period is about 10 minutes, about 15 minutes, about 18 minutes, about 19 minutes, about 20 minutes, about 21 minutes, about 22 minutes, about 23 minutes, about 25 minutes, about 27 minutes, about 30 minutes, and all values in between.

In some embodiments, the method further includes gradually heating the mixture to a fifth temperature. In some embodiments, the fifth temperature is about 80° C., about 90° C., about 95° C., about 98° C., about 99° C., about 100° C., about 101° C., about 102° C., about 105° C., about 110° C., about 115° C., and all values in between.

In some embodiments, the method further includes refluxing the mixture for a fifth time period until the red mixture becomes substantially colorless. In some embodiments, the fifth time period is about 2 minutes, about 5 minutes, about 8 minutes, about 9 minutes, about 10 minutes, about 11 minutes, about 12 minutes, about 15 minutes, about 20 minutes, and all values in between.

In some embodiments, the method further includes cooling the mixture to a sixth temperature. In some embodiments, the sixth temperature is room temperature. In some embodiments, the sixth temperature is about 20° C., about 22° C., about 25° C., about 27° C., about 29° C., about 30° C., and all values in between.

In some embodiments, the method further includes removing solvents by rotary evaporation. In some embodiments, the method further includes adding boiling methanol to the mixture until most of the solid is dissolved.

In some embodiments, the method further includes filtering the mixture through a celite bed under vacuum suction, resulting in a yellow liquid.

In some embodiments, the method further includes adding a silica gel to the yellow liquid. In some embodiments, the method further includes concentrating the mixture to dryness by rotary evaporation. In some embodiments, the method further includes subjecting the mixture to flash column chromatography to yield a yellowish solid. In some embodiments, the yellowish-grey solid includes an aromatic amine.

In some embodiments, the method further includes adding chloroacetamide, bromoacetamide, or iodoacetamide to a mixture of the yellowish solid, sulfur and triethylamine in dimethylformamide In some embodiments, the method further includes stirring the mixture at a seventh temperature for a sixth time period. In some embodiments, the seventh temperature is about 35° C., about 40° C., about 43° C., about 44° C., about 45° C., about 46° C., about 47° C., about 50° C., about 55° C., and all values in between. In some embodiments, the sixth time period is about 10 hours, about 12 hours, about 14 hours, about 15 hours, about 16 hours, about 17 hours, about 18 hours, about 19 hours, about 20 hours, about 22 hours, about 24 hours, about 26 hours, and all values in between.

In some embodiments, the method further includes diluting the mixture with water and extracting with ethyl acetate), wherein an organic layer is produced. In some embodiments, the method further includes drying the organic layer over magnesium sulfate. In some embodiments, the method further includes filtering the mixture. In some embodiments, the method further includes separating the resultant products on silica gel or aluminium oxide (e.g., using flash column chromatography) to yield a yellow solid.

In some embodiments, the method further includes adding the yellow solid into liquid ammonia at an eigth temperature. In some embodiments, the eigth temperature is about −100° C., about −90° C., about −85° C., about −80° C., about −79° C., about −78° C., about −77° C., about −76° C., about −75° C., about −70° C., about −65° C., about -60° C., and all values in between.

In some embodiments, the method further includes stirring the mixture for a seventh time period. In some embodiments, the seventh time period is about 5 minutes, about 10 minutes, about 13 minutes, about 14 minutes, about 15 minutes, about 16 minutes, about 17 minutes, about 20 minutes, about 25 minutes, and all values in between.

In some embodiments, the method further includes adding sodium to the mixture until a blue color remains unchanged for an eighth time period. In some embodiments, the eighth time period is about less than a minute, about 1 minute, about 2 minutes, about 3 minutes, about 4 minutes, about 5 minutes, about 10 minutes, and all values in between.

In some embodiments, the method further includes quenching the reaction by adding ammonium chloride until the blue color substantially disappears. In some embodiments, the method further includes evaporating ammonia under a nitrogen flow at a ninth temperature, resulting in a residue. In some embodiments, the ninth temperature is room temperature. In some embodiments, the ninth temperature is about 20° C., about 22° C., about 25° C., about 27° C., about 29° C., about 30° C., and all values in between.

In some embodiments, the method further includes performing separation by dissolving the residue in an organic solvent such as methanol, ethanol, DMF, and/or the like, followed by addition of silica gel. In some embodiments, the solvent is removed by rotary evaporation to produce a silica gel slurry. In some embodiments, the silica gel slurry is loaded on a silica gel column. In some embodiments, the method further includes eluting out a product including BIA via a gradient of methanol in dichloromethane.

In some embodiments, aspects of the disclosure are directed to a method of attaching BIA to platinum, gold or palladium thereof. As generally disclosed in PCT Publication No. WO2013/151756 (incorporated herein by reference), in some embodiments, a device for identifying one or more molecules (e.g., single molecules) is provided and comprises a first electrode and a second electrode separated from the first electrode by a dielectric material. In some embodiments, at least one of the first electrode and the second electrode includes palladium metal. In some embodiments, the metal is palladium or an alloy of palladium, such as, for example, palladium-platinum or palladium-gold. In some embodiments, at least one reading/trapping molecule is tethered to the first electrode, or to the second electrode, or both. In some embodiments, the reading/trapping molecule is BIA.

Generally, in some embodiments, films including palladium may be prepared by depositing palladium in a layer over a silicon wafer coated with a titanium or chromium adhesion layer. The palladium substrate may then be cut into small pieces (dimension of about 1×1 cm²) and used for preparation of a self-assembled monolayer (SAM). In some embodiments, the palladium substrates can be initially soaked (e.g., in ethanol or isopropanol) and then thoroughly rinsed with ethanol followed by drying. E.g., drying with argon or nitrogen flow. In some embodiments, the clean substrate can be immersed into a solution of BIA, and then subsequently cleaned (e.g., with ethanol) and dried (e.g., with nitrogen flow).

In some embodiments, aspects of the disclosure are directed to a recognition tunneling apparatus for determining and/or sequencing molecules comprising electrodes having bonded thereto 5(6)-mercapto-1H-benzo[d]imidazole-2-carboxamide, or BIA.

As generally disclosed in PCT Publication No. WO2008124706 (incorporated herein by reference), in some embodiments, directed a molecular recognition device that acts as a recognition tunneling apparatus for molecular characterization (e.g., DNA sequencing) through a constriction. The apparatus utilizes electron tunneling current mediated by specific molecular recognition events, such as by, for example, hydrogen-bonding. The recognition tunneling apparatus employs at least one device having at least two sensing electrodes spaced apart by a gap and positions on either side of the constriction. In some embodiments, at least one of the electrodes includes palladium metal. In some embodiments, at least one of the sensing electrodes, or both, has bonded thereto a reading/trapping molecule. In some embodiments, the reading/trapping molecule is BIA.

### EXAMPLES

Synthesis of 5(6)-mercapto-1H-benzo[d]imidazole-2-carboxamide. In some embodiments of the present disclosure, an exemplary process for synthesizing 5(6)-mercapto-1H-benzo[d]imidazole-2-carboxamide (BIA) is provided, the example being illustrated in FIG. 3 and described below. In some embodiments, quantities of the various materials noted below may be substantially the noted amounts, while in other embodiments, may be less or more.

Accordingly, in some embodiments, 2-nitro-4-thiocyanatoaniline (e.g., about 3.51 g, 18 mmol) is added in portions to a stirred solution of potassium hydroxide (e.g., about 6 g) in ethanol (e.g., about 100 ml) at about 5-10° C. and the mixture is stirred for about 30 min at room temperature. An about 25% aqueous solution of sulfuric acid (about 30 ml) is added until the color of the mixture changed from dark violet to bright orange. Solvents may be removed by rotary evaporation. Water (about 200 ml) is added into the mixture, and it is then extracted with ethyl acetate (e.g., about 3×60 ml). The combined organic extracts may then be washed with brine and dried over magnesium sulfate. The solution may then be filtered and the solvent removed by rotary evaporation (for example) to yield compound 1 of FIG. 3 as a red solid (e.g., about 2.94 g, 96%); mp 99-101° C. (reported in literature: 99-101° C.). This may then be used for the next step of synthesis without further purification (for example).

Triethylamine (about 1.22 ml, 8.82 mmol) is added dropwise into a solution of 1 (about 1.0 g, 5.88 mmol) in dichloromethane (about 10 ml) and stirred for about 30 min at room temperature. Benzyl bromide (about 0.84 ml, 7.06 mmol) may then be added into the reaction mixture and the resulting solution stirred for about 42 h at room temperature (for example). The solvent may then be removed by rotary evaporation (for example) and the resulting crude mixture may be dissolved in CH₂Cl₂ (about 100 mL), washed with saturated sodium bicarbonate solution (about 50 mL) and brine (about 20 mL), and dried over MgSO₄. The solution may then be filtered and may be concentrated by a rotary evaporator (for example). The crude product may be purified by flash column chromatography (for example) to furnish compound 2 as a red solid (about 1.07 g, 70%).

4-(Benzylthio)-2-nitrobenzenamine (see reference character 2 in FIG. 3) (about 1.0 g, 3.85 mmol) may then be dissolved in about a 50% aqueous ethanol (about 40 ml), to which a sodium dithionite (about 4.02 g, 23.08 mmol) may be added in portions over a period of about 20 min. The stirred solution may then be gradually heated to about 100° C. and refluxed for about 10 min until the red solution becomes colorless. The solution may then be cooled to room temperature and the solvents removed by rotary evaporation (for example). The crude solid may be extracted with boiling methanol (about 3×50 ml) and filtered through celite bed under vacuum suction to obtain a yellow liquid. Silica gel may be added to the solution, concentrated to dryness, and then subjected to flash column chromatography to yield compound 3 as a yellowish solid (see reference character 3 in FIG. 3) (about 0.73 g, 82%).

Thereafter, chloroacetamide (about 81 mg, 0.87 mmol) may be added to a mixture of compound 3 (see reference character 3 in FIG. 3) (about 200 mg, 0.87 mmol), sulfur (about 111 mg, 3.48 mmol) and Et₃N (about 0.2 mL) in about 2 ml DMF. The mixture may then be stirred at about 45° C. for about 16 h. the solution may then be diluted with water and extracted with ethyl acetate (about 3×30 ml), for example. The combined organic layer may then be dried over magnesium sulfate, and filtered. The products may then be separated with flash column chromatography on silica gel to furnish compound 4 as a yellow solid (see reference character 4 in FIG. 3) (about 98 mg, 40%).

Compound 4 (see reference character 4 in FIG. 3) (about 100 mg, 0.35 mmol) may then be added into liquid ammonia at about -78° C. and stirred for about 15 min. Small pieces of freshly cut sodium may be added into the solution until a blue color remains unchanged for about 3 min and then NH₄Cl may be added until the blue color disappears to quench the reaction Ammonia may be allowed to evaporate under nitrogen flow at room temperature (for example). For separation, the residue may be dissolved in methanol, followed by addition of silica gel. The solvent may then be removed by rotary evaporation (for example) and the silica gel slurry may be loaded on silica gel column. The product (i.e., BIA, as illustrated by see reference character 5 in FIG. 3) may then be eluted out with a gradient of methanol in dichloromethane (about 0 to 5% in about 2 h). Yield: about 47 mg (68%).

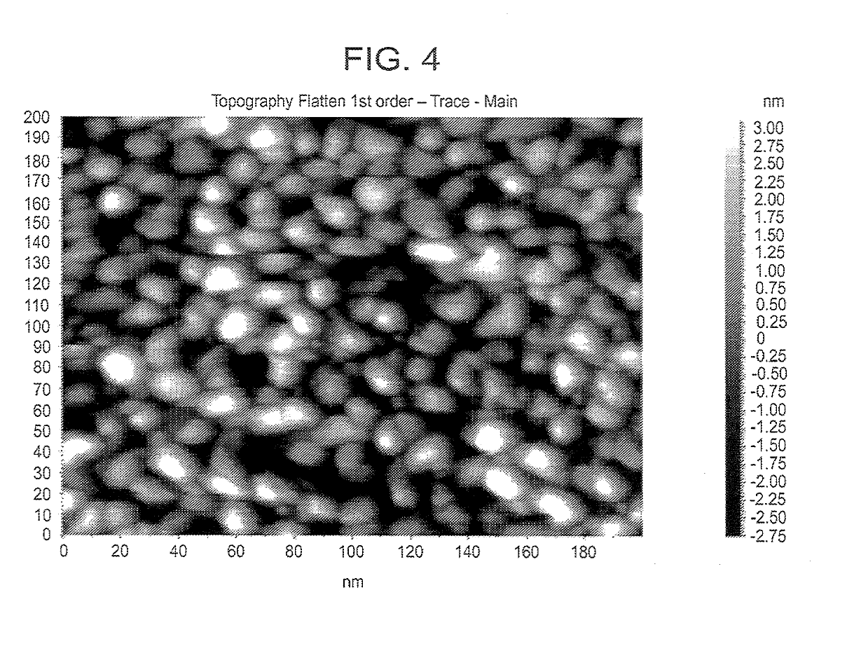

Monolayers of 5(6)-mercapto-1H-benzo[d]imidazole-2-carboxamide on palladium electrodes. In some embodiments, palladium films may be prepared by depositing about 200 nm of palladium in a layer over a silicon wafer coated with about 5 nm thick titanium adhesion layer. The palladium substrate may then be cut into small pieces (dimension of about 1×1 cm²) and used for preparation of self-assembled monolayer (SAM). For example, initially, the substrates are soaked in ethanol and then thoroughly rinsed with ethanol followed by drying with nitrogen flow. The clean substrate may then be immersed into an ethanolic solution (about 0.1-0.5 mM, 2 mL) of 5(6)-mercapto-1H-benzo[d]imidazole-2-carboxamide in a glass vials for about 14-18 hours, and then cleaned with ethanol and dried with a nitrogen flow. In one example, the resultant substrate was imaged by Scanning Tunneling Microscopy (STM) in phosphate buffer at pH 7 (see FIG. 4).

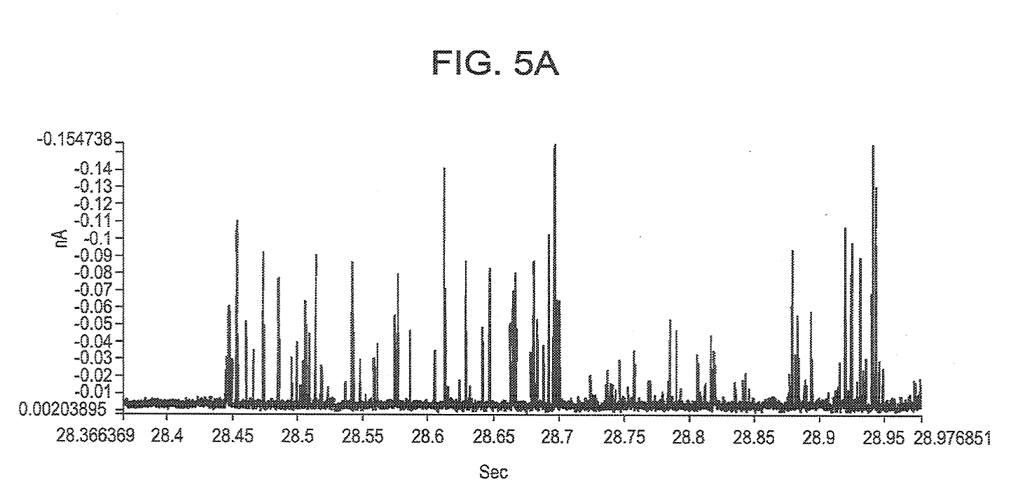

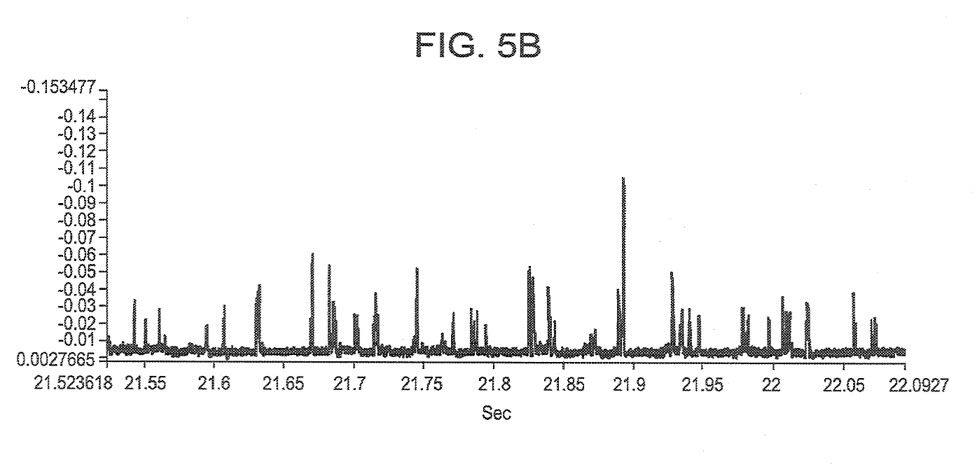

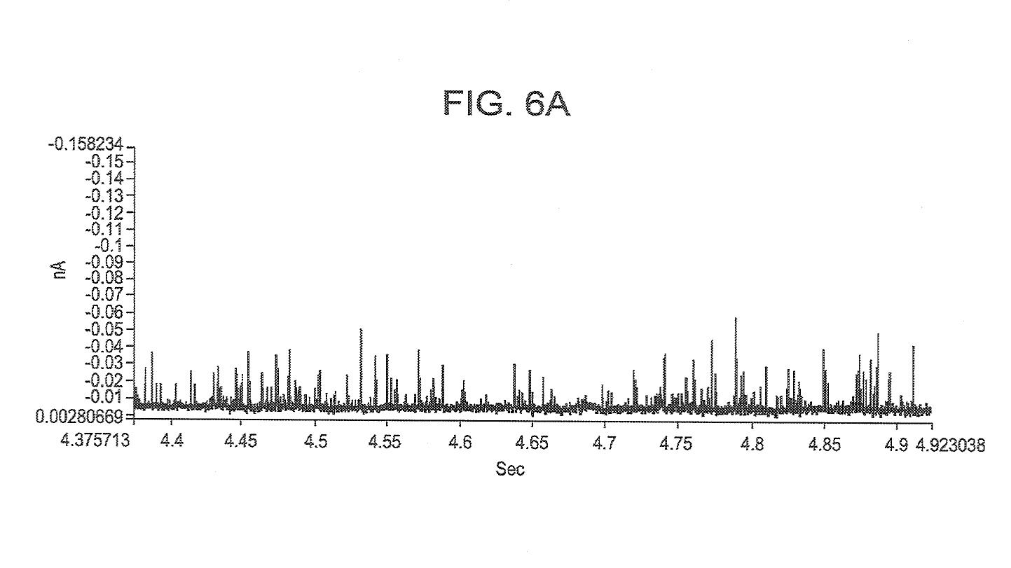

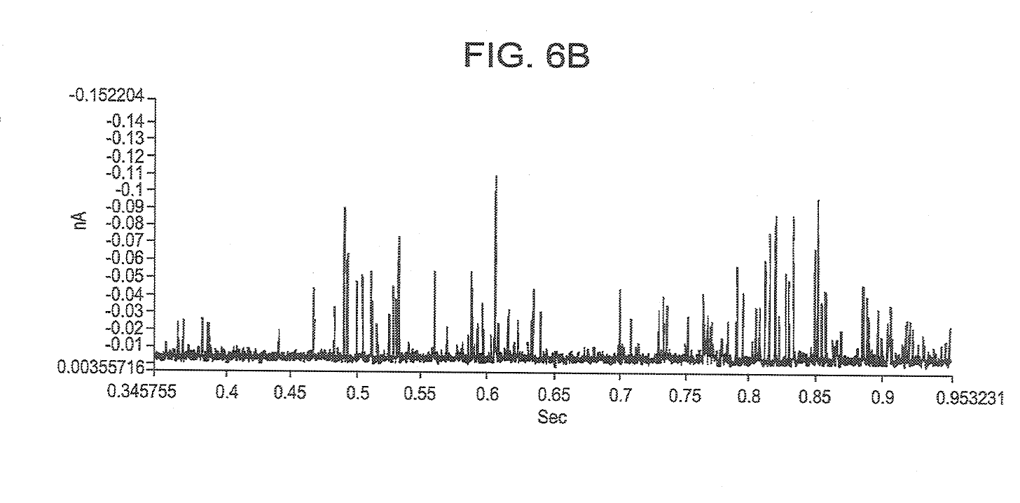

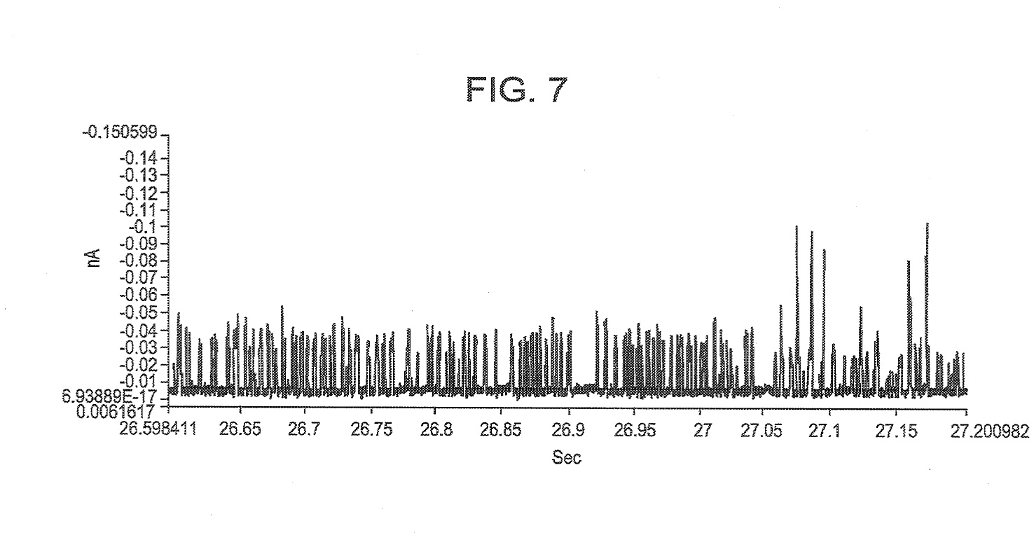

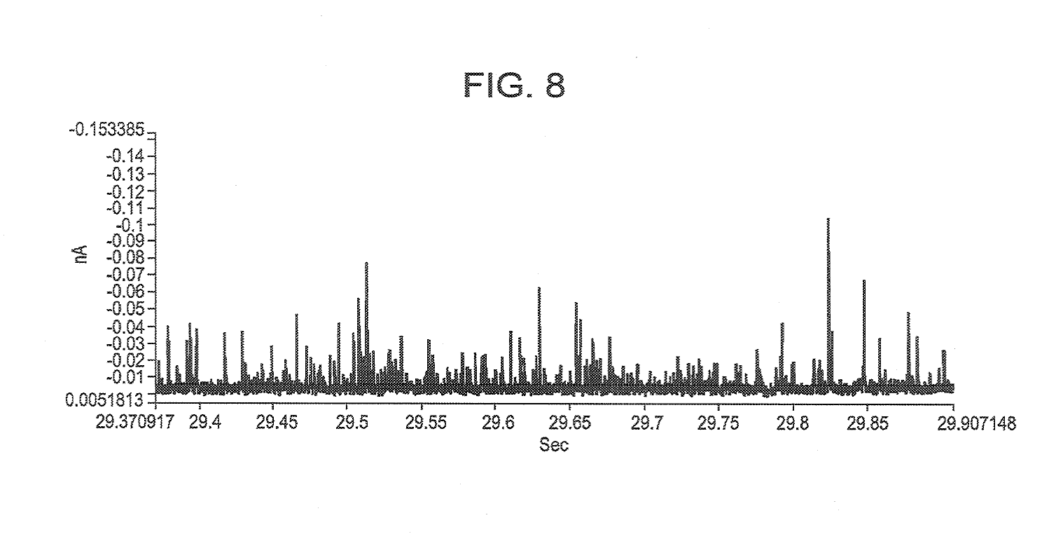

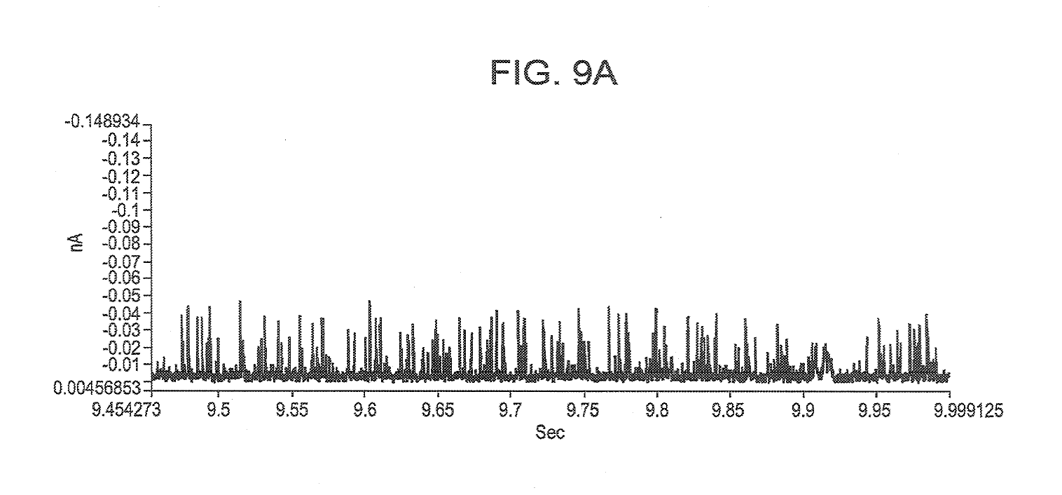

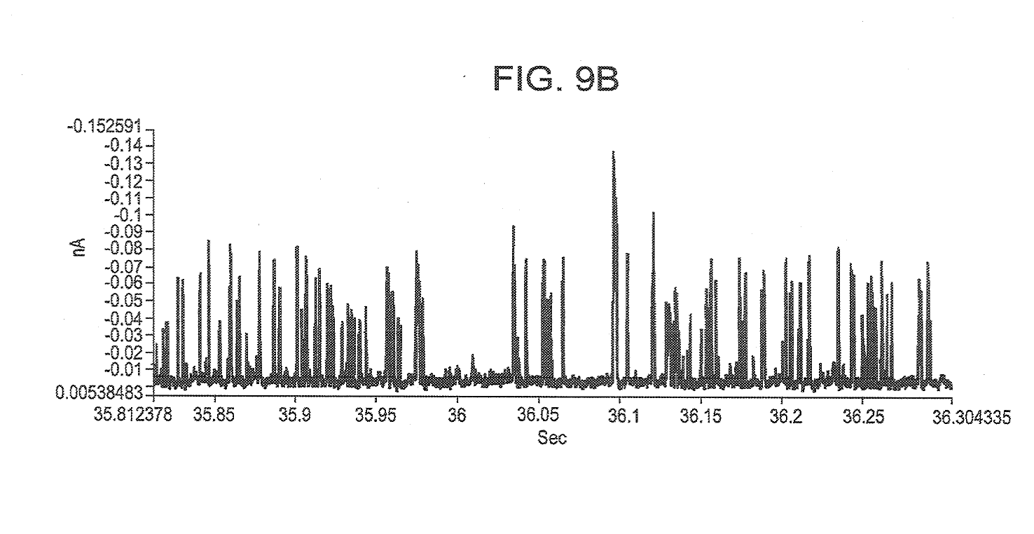

Recognition Tunneling of 5(6)-mercapto-1H-benzo[d]imidazole-2-carboxamide with DNA nucleoside monophosphates. In the some embodiments, 5(6)-mercapto-1H-benzo[d]imidazole-2-carboxamide may be utilized as a reading molecule for measuring, for example, electrical signals of DNA nucleoside monophosphates by recognition tunneling. In such embodiments, two opposed electrodes are spaced apart by a gap of about 2.5 nm (with a set point of about 0.5 v bias and about 4 pA background tunneling current, for example). Each electrode may be functionalized with 5(6)-mercapto-1H-benzo[d]imidazole-2-carboxamide that is chemically-bonded to the electrodes, and forms non-covalent bonds with the target molecule. Accordingly, for example, each DNA nucleoside monophosphates is dissolved with a concentration of about 100 μM in an about 1.0 mM phosphate buffer, of a pH of about 7. Examples of recognition tunneling signals generated for each nucleotide are shown in FIGS. 5, 6, 7 and 8. Accordingly, as shown, the amplitude of these signals are larger (and in some embodiments, considerably larger) than signals produced by an earlier universal reader molecule, 4(5)-(2-mercaptoethyl)-1H-imidazole-2-carboxamide. In addition, in some embodiments, the replacement of the ethylene linker by a phenol ring adds hydrophobic character to the molecule.

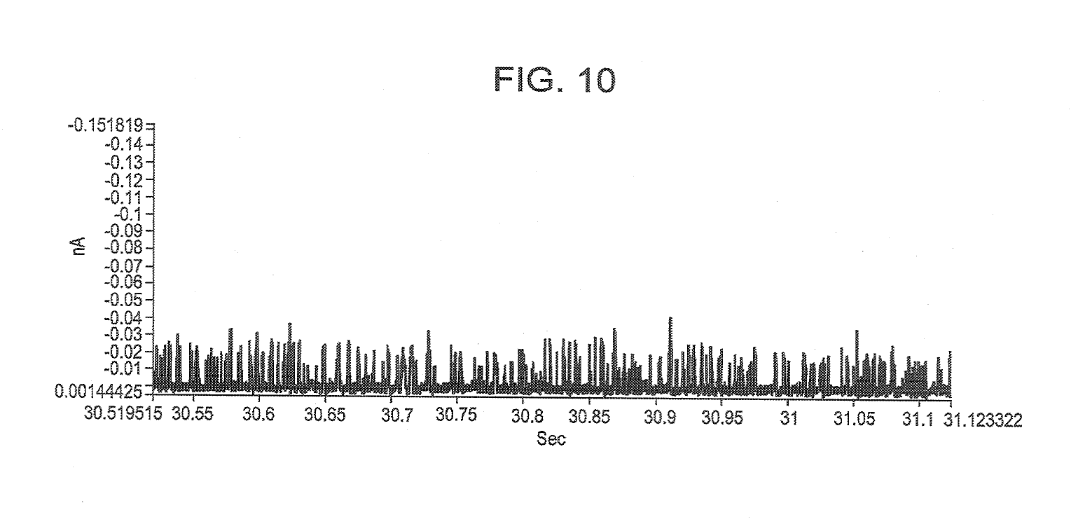

Recognition Tunneling of 5(6)-mercapto-1H-benzo[d]imidazole-2-carboxamide with L-glycine. In some embodiments, an amino acid can also be trapped in the nanogap by reader molecules, and generate distinguishable tunneling signals. FIG. 10 show a tunneling spectrum generated by glycine, for example.

### SVM analysis of 4(5)-(2-mercaptoethyl)-1H-imidazole-2-carboxamide (ICA) & 5(6)-mercapto-1H-benzo[d]imidazole-2-carboxamide BIA) reader at about 2 pA and about 4 pA set point

Support vector machines (SVMs)—a machine learning algorithm—are employed to analyze data from the recognition tunneling measurements. Different features of the tunnel current spikes were used to discriminate different DNA nucleoside monophosphates (Table 1).

TABLE 1
List of parameters used in SVM analysis

| Number | Parameter |
|---|---|
| 1 | Peak top average |
| 2 | Peak width |
| 3 | Peak roughness |
| 4 | Peak total power |
| 5 | Peak iFFT low |
| 6 | Peak iFFT medium |
| 7 | Peak iFFT high |
| 8 | Peak frequency |
| 9 | Peak FFT1 |
| 10 | Peak FFT2 |
| 11 | Peak FFT3 |
| 12 | Peak FFT4 |
| 13 | Peak FFT5 |
| 14 | Peak FFT6 |
| 15 | Peak FFT7 |
| 16 | Peak FFT8 |
| 17 | Peak FFT9 |
| 18 | Peak FFT10 |
| 19 | Peak high low ratio |
| 20 | Peak Odd FFT |
| 21 | Peak Even FFT |
| 22 | Peak Odd Even Ratio |
| 23 | Peaks In Cluster |
| 24 | Cluster frequency |
| 25 | Cluster average Amplitude |
| 26 | Cluster top Average |
| 27 | Cluster Width |
| 28 | Cluster roughness |
| 29 | Cluster max amplitude |
| 30 | Cluster total power |
| 31 | Cluster iFFT low |
| 32 | Cluster iFFT medium |
| 33 | Cluster iFFT high |
| 34 | Cluster FFT1 |
| 35 | Cluster FFT2 |
| 36 | Cluster FFT3 |
| 37 | Cluster FFT4 |
| 38 | Cluster FFT5 |
| 39 | Cluster FFT6 |
| 40 | Cluster FFT7 |
| 41 | Cluster FFT8 |
| 42 | Cluster FFT9 |
| 43 | Cluster FFT10 |
| 44 | Cluster FFT11 |
| 45 | Cluster FFT12 |
| 46 | Cluster FFT13 |
| 47 | Cluster FFT14 |
| 48 | Cluster FFT15 |
| 49 | Cluster FFT16 |
| 50 | Cluster FFT17 |
| 51 | Cluster FFT18 |
| 52 | Cluster FFT19 |
| 53 | Cluster FFT20 |
| 54 | Cluster FFT21 |
| 55 | Cluster FFT22 |
| 56 | Cluster FFT23 |
| 57 | Cluster FFT24 |
| 58 | Cluster FFT25 |
| 59 | Cluster FFT26 |
| 60 | Cluster FFT27 |
| 61 | Cluster FFT28 |
| 62 | Cluster FFT29 |
| 63 | Cluster FFT30 |
| 64 | Cluster FFT31 |
| 65 | Cluster FFT32 |
| 67 | Cluster FFT33 |
| 68 | Cluster FFT34 |
| 69 | Cluster FFT35 |
| 70 | Cluster FFT36 |
| 71 | Cluster FFT37 |
| 72 | Cluster FFT38 |
| 73 | Cluster FFT39 |
| 74 | Cluster FFT40 |
| 75 | Cluster FFT41 |
| 76 | Cluster FFT42 |
| 77 | Cluster FFT43 |
| 78 | Cluster FFT44 |
| 79 | Cluster FFT45 |
| 80 | Cluster FFT46 |
| 81 | Cluster FFT47 |
| 82 | Cluster FFT48 |
| 83 | Cluster FFT49 |
| 84 | Cluster FFT50 |
| 85 | Cluster FFT51 |
| 86 | Cluster FFT52 |
| 87 | Cluster FFT53 |
| 88 | Cluster FFT54 |
| 89 | Cluster FFT55 |
| 90 | Cluster FFT56 |
| 91 | Cluster FFT57 |
| 92 | Cluster FFT58 |
| 93 | Cluster FFT59 |
| 94 | Cluster FFT60 |
| 95 | Cluster high low |
| 96 | Cluster_freq_Maximum_Peaks1 |
| 97 | Cluster_freq_Maximum_Peaks2 |
| 98 | Cluster_freq_Maximum_Peaks3 |
| 99 | Cluster_freq_Maximum_Peaks4 |
| 100 | Cluster Cepstrum1 |
| 101 | Cluster Cepstrum2 |
| 102 | Cluster Cepstrum3 |
| 103 | Cluster Cepstrum4 |
| 104 | Cluster Cepstrum5 |
| 105 | Cluster Cepstrum6 |
| 106 | Cluster Cepstrum7 |
| 107 | Cluster Cepstrum8 |
| 108 | Cluster Cepstrum9 |
| 109 | Cluster Cepstrum10 |
| 110 | Cluster Cepstrum11 |
| 111 | Cluster Cepstrum12 |
| 112 | Cluster Cepstrum13 |
| 113 | Cluster Cepstrum14 |
| 114 | Cluster Cepstrum15 |
| 115 | Cluster Cepstrum16 |
| 116 | Cluster Cepstrum17 |
| 117 | Cluster Cepstrum18 |
| 118 | Cluster Cepstrum19 |
| 119 | Cluster Cepstrum20 |
| 120 | Cluster Cepstrum21 |
| 121 | Cluster Cepstrum22 |
| 122 | Cluster Cepstrum23 |
| 123 | Cluster Cepstrum24 |
| 124 | Cluster Cepstrum25 |
| 125 | Cluster Cepstrum26 |
| 126 | Cluster Cepstrum27 |
| 127 | Cluster Cepstrum28 |
| 128 | Cluster Cepstrum29 |
| 129 | Cluster Cepstrum30 |
| 130 | Cluster Cepstrum31 |
| 131 | Cluster Cepstrum32 |
| 132 | Cluster Cepstrum33 |
| 133 | Cluster Cepstrum34 |
| 134 | Cluster Cepstrum35 |
| 135 | Cluster Cepstrum36 |
| 136 | Cluster Cepstrum37 |
| 137 | Cluster Cepstrum38 |
| 138 | Cluster Cepstrum39 |
| 139 | Cluster Cepstrum40 |
| 140 | Cluster Cepstrum41 |
| 141 | Cluster Cepstrum42 |
| 142 | Cluster Cepstrum43 |
| 143 | Cluster Cepstrum44 |
| 144 | Cluster Cepstrum45 |
| 145 | Cluster Cepstrum46 |
| 146 | Cluster Cepstrum47 |
| 147 | Cluster Cepstrum48 |
| 148 | Cluster Cepstrum49 |
| 149 | Cluster Cepstrum50 |
| 150 | Cluster Cepstrum51 |
| 151 | Cluster Cepstrum52 |
| 152 | Cluster Cepstrum53 |
| 153 | Cluster Cepstrum54 |
| 154 | Cluster Cepstrum55 |
| 155 | Cluster Cepstrum56 |
| 156 | Cluster Cepstrum57 |
| 157 | Cluster Cepstrum58 |
| 158 | Cluster Cepstrum59 |
| 159 | Cluster Cepstrum60 |
| 160 | Cluster Cepstrum61 |

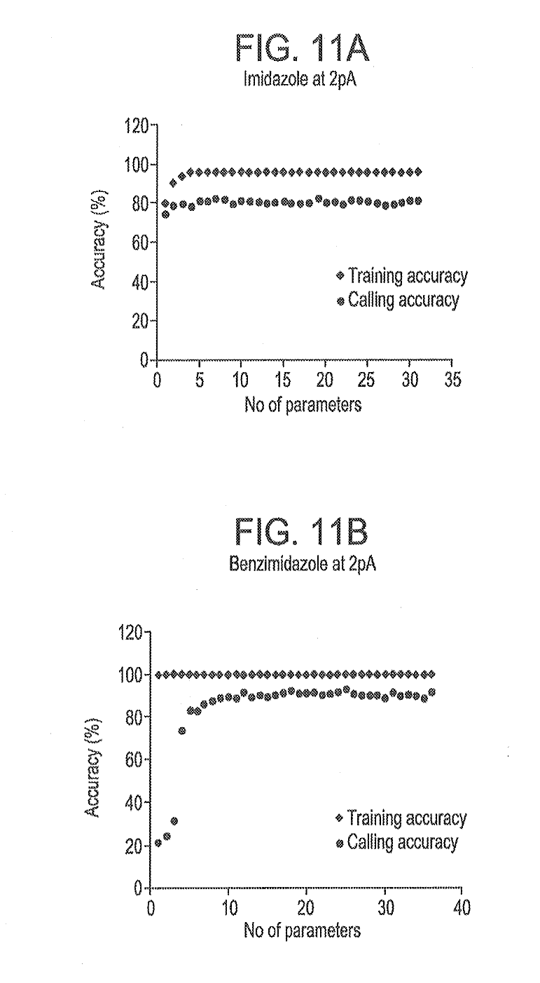

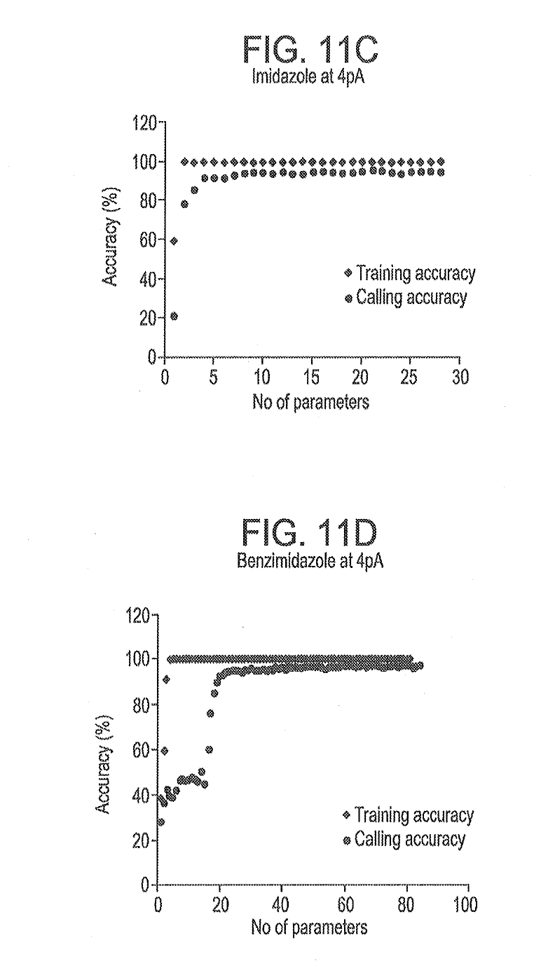

In the initial step of the analysis process, a sub-set of data was used to train the SVM. Then the rest of the data was feed to the trained SVM and correct identification of all the different DNA monophosphates was obtained with a significant level of accuracy. All the current spikes having amplitude under 15 pA were discarded during data filtering. These spikes originated from water molecules. The presence of such low amplitude spikes in the control experiments with phosphate buffer justified their origin and hence our data filtering. Some common spikes were found in case of different DNA monophosphates and were also discarded during the data filtering process. Approximately 35-40% data was discarded in this process. The rest of the tunnel current spikes were classified under five classes (deoxyadenosine monophosphate, deoxycytidine monophosphate, deoxyguanosine monophosphate, deoxythymidine monophosphate and deoxymethyl-cytidine monophisphate). All the tunneling measurements were done at two different set point (about 2 pA and about 4 pA). Based on results from the SVM analysis (see FIGS. 11A-11D), FIG. 11A shows the ability of ICA to separate different DNA monophosphates at 2 pA set point. The horizontal axis represents the number of parameter sets that are being introduced during SVM analysis. Generally, each parameter set contains 2 to 4 parameters. The vertical axis represents the percentage of training accuracy of the SVM (solid squares) and calling accuracy of the trained SVM (solid circles). FIGS. 11B-11D correspond to BIA at 2 pA set point, ICA at 4 pA set point and BIA at 4 pA set point, respectively. Under both conditions, the BIA reader proves to be a better choice over ICA reader as the former shows better calling accuracy than the latter (summarized in Table 2).

TABLE 2

| | Calling accuracy at 2 pA | Calling accuracy at 4 pA |
|---|---|---|
| Reader | (average top 3 value) | (average top 3 value) |
| ICA | 85.70 | 95.38 |
| BIA | 91.92 | 97.67 |

In some embodiments, aspects of the disclosure are directed to a compound for trapping and reading an analyte in a tunnel junction, the compound having a structure comprising a five membered aromatic ring fused either with a conductive ring moiety, or a derivative of the conductive ring moiety, thereby enhancing the conductivity of the compound. In some embodiments, the compound is 5(6)-mercapto-1H-benzo[d]imidazole-2-carboxamide. In some embodiments, aspects of the disclosure are directed to a composition comprising the compound(s) disclosed herein.

In some embodiments, aspects of the disclosure are directed to a compound of a reader molecule for use in a recognition tunneling apparatus, the reader molecule comprising a fusion of an aromatic ring with a heterocycle. The reader molecule forms complexes with biochemical molecules through non-covalent interactions, the interactions comprising at least one of hydrogen bonding, aromatic interactions, stacking interaction, and hydrophobic interactions. As a result of the formed complexes, tunneling current signals are generated in the recognition tunneling apparatus. In some embodiments, the compound is 5(6)-mercapto-1H-benzo[d]imidazole-2-carboxamide. In some embodiments, aspects of the disclosure are directed to a composition comprising the compound(s) disclosed herein.

In some embodiments, aspects of the disclosure are directed to a compound of the formula C₈H₇N₃OS, having a structure given by the structure illustrated in FIG. 1B. In some embodiments, aspects of the disclosure are directed to a composition comprising the compound(s) disclosed herein.

In some embodiments, aspects of the disclosure are directed to a method of synthesizing 5(6)-mercapto-1H-benzo[d]imidazole-2-carboxamide. The method includes forming a doubly amintated phenol ring, and treating the doubly amintated phenol ring with chloroacetamide, wherein as a result of treating, a fusion of an aromatic ring with a heterocycle is formed.

In some embodiments, aspects of the disclosure are directed to a method for synthesizing 5(6)-mercapto-1H-benzo[d]imidazole-2-carboxamide. The method includes:

(a) adding 2-Nitro-4-thiocyanatoaniline in portions to a stirred solution of potassium hydroxide in ethanol at a first temperature, and stirring the mixture for a first period of time at a second temperature;

(b) adding an aqueous solution of sulfuric acid to the mixture obtained in step (a) until the color of the mixture changes from a first color to a second color;

(c) removing one or more solvents from the mixture obtained in step (b);

(d) adding water to the mixture obtained in step (c) and then extracting water with ethyl acetate, to produce an organic extract from the mixture obtained in step (c);

(e) washing the organic extract obtained in step (d) with brine;

(f) drying the washed organic extract obtained in step (e) over magnesium sulfate;

(g) filtering the mixture obtained in step (f); and

(h) removing the solvent from the mixture obtained in step (g), wherein a red solid is produced.

In some embodiments, the method further includes:

(i) adding triethylamine dropwise into the mixture obtained in step (h) in dichloromethane;

(j) stirring the mixture obtained in step (i) for a second period of time at a third temperature;

(k) adding benzyl bromide to the mixture obtained in step (j);

(l) stirring the mixture obtained in step (k) for a third period of time at a fourth temperature;

(m) removing solvent from the mixture obtained in step (1) resulting in a crude oil;

(n) dissolving the crude oil obtained in step (m) in CH₂Cl₂ the CH₂Cl₂ being washed with at least one of saturated sodium bicarbonate solution and brine;

(o) drying the crude oil obtained in step (n) over MgSO₄,

(p) filtering the mixture obtained in step (o);

(q) concentrating the mixture obtained in step (p); and

(r) purifying the mixture obtained in step (q).

In some embodiments, the concentrating is performed via rotary evaporation. In some embodiments, the purifying is performed via flash column chromatography.

In some embodiments, the method further includes:

(s) dissolving the mixture obtained in step (r) an aqueous ethanol;

(t) adding sodium dithionite in portions to the mixture obtained in step (s) over a fourth time period;

(u) gradually heating the mixture obtained in step (t) to a fifth temperature;

(v) refluxing the mixture obtained in step (v) for a fifth time period until the red mixture becomes colorless;

(w) cooling the mixture obtained in step (v) to a sixth temperature;

(x) removing solvents from the mixture obtained in step (w);

(y) extracting solids from the mixture obtained in step (x) via boiling methanol;

(z) filtering the mixture obtained in step (z) through a celite bed under vacuum suction, resulting in a yellow liquid;

(aa) adding a silica gel to the mixture obtained in step (z);

(bb) concentrating the mixture obtained in step (aa) to dryness; and

(cc) subjecting the mixture obtained in step (bb) to flash column chromatography to yield a yellowish-grey solid.

In some embodiments, the method further includes:

(dd) adding chloroacetamide to a mixture of the solid obtained in step (cc), sulfur and Et₃N (about 0.2 mL) in DMF;

(ee) stirring the mixture obtained in step (dd) at a seventh temperature for a sixth time period;

(ff) diluting the mixture obtained in step (ee) with water and extracting the water with ethyl acetate, wherein an organic layer is produced;

(gg) drying the organic layer obtained in step (ff) over magnesium sulfate;

(hh) filtering the mixture obtained in step (gg) under reduced pressure;

(ii) separating resultant products obtained in step (hh) on silica gel to yield a yellow solid.

In some embodiments, the method further includes:

(jj) adding the yellow solid obtained in step (ii) into liquid ammonia at an eighth temperature;

(kk) stirring the mixture obtained in step (jj) for a seventh time period

(ll) adding sodium to the mixture obtained in step (kk) until a blue color remains unchanged for anan eighth time period;

(mm) quenching the reaction in step (ll) by adding NH₄Cl to the mixture obtained in step (ll) until the blue color disappears; and

(nn) evaporating ammonia from the mixture obtained in step (mm) under a nitrogen flow at a ninth temperature resulting in a residue.

In some embodiments, the method further includes:

(oo) performing separation by dissolving the residue obtained in step (nn) in methanol, followed by addition of silica gel.

In some embodiments, the method further includes:

(pp) removing solvent from the mixture obtained in step (oo) by rotary evaporation to produce a silica gel slurry.

In some embodiments, the method further includes:

(qq) loading the silica gel slurry obtained in step (pp) on a silica gel column.

In some embodiments, the method further includes eluting out 5(6)-mercapto-1H-benzo[d]imidazole-2-carboxamide from any mixture disclosed herein via a gradient of methanol in dichloromethane.

In some embodiments, aspects of the disclosure are directed to a method of attaching 5(6)-mercapto-1H-benzo[d]imidazole-2-carboxamide to platinum, gold or palladium, the method activating the thiol moiety thereof.

In some embodiments, aspects of the disclosure are directed to a recognition tunneling apparatus for determining and/or sequencing molecules, the apparatus comprising electrodes having bonded thereto 5(6)-mercapto-1H-benzo[d]imidazole-2-carboxamide.

Any and all references to publications or other documents, including but not limited to, patents, patent applications, articles, webpages, books, etc., presented anywhere in the present application, are herein incorporated by reference in their entirety.

Although example embodiments of the devices, systems and methods have been described herein, other modifications are possible. As noted elsewhere, these embodiments have been described for illustrative purposes only and are not limiting. Other embodiments are possible and are covered by the disclosure, which will be apparent from the teachings contained herein. Thus, the breadth and scope of the disclosure should not be limited by any of the above-described embodiments but should be defined only in accordance with any and all claims supported by the present disclosure and their equivalents. In addition, any logic flow depicted in the above disclosure and/or accompanying figures may not require the particular order shown, or sequential order, to achieve desirable results.

Moreover, embodiments of the subject disclosure may include methods, systems and devices which may further include any and all elements from any other disclosed methods, systems, and devices. In other words, elements from one or another disclosed embodiments may be interchangeable with elements from other disclosed embodiments. In addition, one or more features/elements of disclosed embodiments may be removed and still result in patentable subject matter (and thus, resulting in yet more embodiments of the subject disclosure). Still further, some embodiments of the present disclosure may be distinguishable from prior art on the basis of specific lack of one or more features/elements (i.e., claims directed toward such embodiments include negative limitations to distinguish over the prior art). Other implementations of some of the embodiments disclosed herein may be within the scope of at least some of the following claims.

1. A compound for trapping and reading an analyte in a tunnel junction, the compound having a structure comprising a five membered aromatic ring fused either with a conductive ring moiety, or a derivative of the conductive ring moiety, thereby enhancing the conductivity of the compound.

2. The compound according to claim 1, wherein the compound is 5(6)-mercapto-1H-benzo[d]imidazole-2-carboxamide.

3. A composition comprising the compound of claim 1.

4. A compound of a reader molecule for use in a recognition tunneling apparatus, the reader molecule comprising a fusion of an aromatic ring with a heterocycle, wherein the reader molecule forms complexes with biochemical molecules through non-covalent interactions, the interactions comprising at least one of hydrogen bonding, aromatic interactions, stacking interaction, and hydrophobic interactions, wherein as a result of the formed complexes, tunneling current signals are generated in the recognition tunneling apparatus.

5. The compound according to claim 4, wherein the compound is 5(6)-mercapto-1H-benzo[d]imidazole-2-carboxamide.

6. A composition comprising the molecule of claim 4.

7. A compound of the formula C₈H₇N₃OS, having a structure given by:

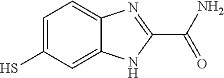 [SMILES: NC(=O)c1nc2ccc(S)cc2[nH]1]

8. A composition comprising the compound of claim 7.

9-22. (canceled)
